(19) United States
(12) Patent Application Publication
(10) Pub. No.: US 2019/0192458 A1
(43) Pub. Date: Jun. 27, 2019

(54) THERAPEUTIC/PROPHYLACTIC AGENT FOR GRAFT-VERSUS-HOST DISEASE, FIBROCYTE INVASION INHIBITOR, AND INHIBITOR AGAINST TEAR REDUCTION AND REDUCTION IN GOBLET CELLS

(71) Applicant: KEIO UNIVERSITY, Minato-ku Tokyo (JP)

(72) Inventors: Shin MUKAI, Shinjuku-ku Tokyo (JP); Yoko OGAWA, Shinjuku-ku Tokyo (JP); Kazuo TSUBOTA, Shinjuku-ku Tokyo (JP); Yutaka KAWAKAMI, Shinjuku-ku Tokyo (JP)

(21) Appl. No.: 16/090,933

(22) PCT Filed: Apr. 5, 2017
(86) PCT No.: PCT/JP2017/014290

§ 371 Date: Feb. 1, 2019

Related U.S. Application Data
(60) Provisional application No. 62/318,404, filed on Apr. 5, 2016.

(51) Int. Cl. A61K 31/192; A61P 37/06

(52) U.S. Cl. CPC A61K 31/192; A61P 37/06

(57) ABSTRACT

Provided are a novel therapeutic or prophylactic agent for graft-versus-host disease, a novel agent for inhibiting fibrocyte infiltration, and a novel agent for inhibiting a decrease in tear secretion and a decrease of goblet cells. A method of treating or preventing graft-versus-host disease comprises administering a phenylbutyric acid or a pharmacologically acceptable salt thereof to a patient in need of treatment or prevention of graft-versus-host disease. It is preferable that the graft-versus-host disease be a graft-versus-host disease that manifests after bone marrow transplantation. It is preferable that the graft-versus-host disease be ocular graft-versus-host disease.

### CROSS REFERENCE TO RELATED APPLICATION

This is the Non-Provisional application based upon the Provisional Application No. 62,318404, filed on Apr. 5, 2016. Provisional Application No. 62,318404, filed on Apr. 5, 2016, claims the benefit of the National Stage Application No. PCT/JP2017/014290, filed on Apr. 5, 2017, the disclosure of which is also incorporated herein by reference.

### TECHNICAL FIELD

The present invention relates to a therapeutic or prophylactic agent for graft-versus-host disease, an agent for inhibiting fibrocyte infiltration, and an agent for inhibiting a decrease in tear secretion and a decrease of goblet cells.

### BACKGROUND ART

Some hematologic malignancies resulting from tumorigenesis of hematopoietic stem cells are treated with hematopoietic stem cell transplantation, which is an established therapy involving transplantation of healthy, allogeneic hematopoietic stem cells with a goal of radical cure of leukemia. As recent reports show, a donor's graft such as bone marrow cells, peripheral blood, and cord blood induces immune response that targets and attacks organs of a recipient (graft-versus-host disease, GVHD).

As an example of therapeutic agents for graft-versus-host disease, rebamipide and diquafosol are known as treatment of the ocular surface. Non-patent Document 1 discloses a combined use of rebamipide and diquafosol for alleviating dry eye, which is a symptom of graft-versus-host disease.

In particular, chronic graft-versus-host disease (cGVHD) can be a serious complication after hematopoietic stem cell transplantation (bone marrow transplantation), and even be life-threatening for some patients. Although cGVHD has been studied intensively over the past decades, an effective therapy for cGVHD remains to be developed. Patients with cGVHD are commonly treated with immunosuppressants such as ciclosporin and steroids. However, these therapies tend to provide unsatisfactory outcomes. In view of the above circumstances, there have been strong clinical demands for effective methods of treating cGVHD. For example, Non-Patent Document 2 discloses that cellular senescence of donor immune cells and recipient cells is adversely involved in the development process of cGVHD.

Meanwhile, the phenomenon “endoplasmic reticulum stress” is known to play an adverse role in the manifestation of chronic inflammation and age-related diseases (for example, Non-Patent Documents 3 and 4). Below, endoplasmic reticulum stress will be overviewed with reference to FIG. 9 and FIG. 10. When a protein is synthesized in the endoplasmic reticulum, the protein is expected to be folded correctly. This may be assisted by an endoplasmic reticulum chaperone. However, correct folding of a protein in the endoplasmic reticulum may be disturbed by hypoxia, calcium ion deficiency, oxidative injuries, virus infection, and inflammatory cytokines.

As shown in FIG. 9, accumulation of unfolded and misfolded proteins in the endoplasmic reticulum may induce endoplasmic reticulum stress. This, in turn, may activate 3 transmembrane proteins (inositol-requesting (IRE) 1α, PKR-like ER kinase (PERK), and activating transcription factor (ATF) 6α) to initiate a response to unfolded proteins (UPR). Prolonged or unsuccessful UPR may, however, result in inflammation and activation of the apoptosis pathway (Non-Patent Document 5). As a result, unsuccessful UPR is known to induce expression of: (1) Thioredoxin-interacting protein (TXNIP), a proinflammatory molecule, and Nuclear factor κ-light chain enhancer (NF-κB), a transcription factor of activated B cells; and (2) C/EBP homologous protein (CHOP), an apoptosis protein (Non-Patent Documents 6 to 8).

However, the correlation of endoplasmic reticulum stress with GVHD (in particular with cGVHD) has not been known until now.

Non-Patent Document 1: Mio Yamane, Yoko Ogawa,et al., “Long-term Rebamipide and Diquafosol in Two Cases of Immune-Mediated Dry Eye” Optometry and Vision Science, Vol. 92, No. 4S, April 2015

Non-Patent Document 2: Kawai M, Ogawa Y, Shimmura S, et al. Expression and localization of aging markers in lacrimal gland of chronic graft-versus-host disease. Scientific Reports. 2013; 3(2445):1-6.

Non-Patent Document 3: Brown M K, NirinjiniNaidoo. The endoplasmic reticulum stress response in aging and age-related diseases. Front Physiol. 2012; 3(263):1-10.

Non-Patent Document 4: Garg A D, Kaczmarek A, Krysko O, Vandenabeele P, Krysko D V, Agostinis P. ER stress-induced inflammation: does it aid or impede disease progression? Trends Mol Med. 2012; 18:589-598.

Non-Patent Document 5: Lee A S. The ER chaperone and signaling regulator GRP78/BiP as a monitor of endoplasmic reticulum stress. Methods. 2005; 35:373-381.

Non-Patent Document 6: Anthony T G, Wek R C. TXNIP Switches Tracks toward a Terminal UPR. Cell Metab. 2012; 16:135-137.

Non-Patent Document 7: Lerner A G, Upton J P, Praveen P V K, et al. IRE1a Induces Thioredoxin-Interacting Protein to Activate the NLRP3 Inflammasome and Promote Programmed Cell Death under Irremediable ER Stress. Cell Metab. 2012; 16:250-264.

Non-Patent Document 8: Oslowski C M, Hara T, Murphy B O S, et al. Thioredoxin-Interacting Protein Mediates ER Stress-Induced Beta Cell Death through Initiation of the Inflammasome. Cell Metab. 2012; 16:265-273.

### DISCLOSURE OF THE INVENTION

### Problems to be Solved by the Invention

The present invention has been devised based on the above circumstances, and an object of the present invention is to provide a novel therapeutic or prophylactic agent for graft-versus-host disease, a novel agent for inhibiting fibrocyte infiltration, and a novel agent for inhibiting a decrease in tear secretion and a decrease of goblet cells.

### Means for Solving the Problems

The inventors of the present invention have found that graft-versus-host disease is accompanied by fibrocyte infiltration in a lacrimal gland, and this fibrocyte infiltration is inhibited by a phenylbutyric acid or a pharmacologically acceptable salt thereof. Further, the inventors of the present invention have reached a novel hypothesis in that (1) a cGVHD-affected organ may show an increased level of endoplasmic reticulum stress, and (2) alleviation of endoplasmic reticulum stress can be a potent therapeutic method of alleviating a serious symptom resulting from cGVHD (in particular systemic cGVHD). Thus, the present invention has now been completed. That is, the embodiments of the present invention include the following.
<1> A therapeutic or prophylactic agent for graft-versus-host disease, comprising a phenylbutyric acid or a pharmaceutically acceptable salt thereof as an active ingredient.
<2> The therapeutic or prophylactic agent according to <1>, wherein the graft-versus-host disease is a graft-versus-host disease that manifests after bone marrow transplantation.
<3> The therapeutic or prophylactic agent according to <1> or <2>, wherein the graft-versus-host disease is ocular graft-versus-host disease.
<4> The therapeutic or prophylactic agent according to <3>, wherein the graft-versus-host disease is dry eye.
<5> The therapeutic or prophylactic agent according to any one of <1> to <4>, wherein the graft-versus-host disease is a graft-versus-host disease accompanied by fibrocyte infiltration. [0012]
<6> The therapeutic or prophylactic agent according to any one of <1> to <5>, wherein the phenylbutyric acid is 4-phenyl-n-butyric acid or a pharmacologically acceptable salt thereof.
<7> An agent for inhibiting fibrocyte infiltration, comprising a phenylbutyric acid or a pharmaceutically acceptable salt thereof as an active ingredient.
<8> The agent for inhibiting fibrocyte infiltration according to <7>, wherein the phenylbutyric acid is 4-phenyl-n-butyric acid or a pharmacologically acceptable salt thereof.
<9> An agent for inhibiting a decrease in goblet cells, comprising a phenylbutyric acid or a pharmaceutically acceptable salt thereof as an active ingredient.
<10> The agent for inhibiting a decrease in goblet cells according to <9>, wherein the phenylbutyric acid is 4-phenyl-n-butyric acid or a pharmacologically acceptable salt thereof.
<11> An agent for inhibiting a decrease in tear secretion, comprising a phenylbutyric acid or a pharmaceutically acceptable salt thereof as an active ingredient.
<12> The agent for inhibiting a decrease in tear secretion according to <11>, wherein the phenylbutyric acid is 4-phenyl-n-butyric acid or a pharmacologically acceptable salt thereof.

The following is also preferred as the embodiment of the present invention.
(1) A method of treating or preventing graft-versus-host disease, the method comprising administering a phenylbutyric acid or a pharmacologically acceptable salt thereof.
(2) The method according to (1), wherein the graft-versus-host disease is a graft-versus-host disease that manifests after bone marrow transplantation.
(3) The method according to (1) or (2), wherein the graft-versus-host disease is ocular graft-versus-host disease.
(4) The method according to (3), wherein the graft-versus-host disease is dry eye.
(5) The method according to any one of (1) to (4), wherein the graft-versus-host disease is a graft-versus-host disease accompanied by fibrocyte infiltration.
(6) The method according to any one of (1) to (5), wherein the phenylbutyric acid is 4-phenyl-n-butyric acid or a pharmacologically acceptable salt thereof.
(7) A method of inhibiting fibrocyte infiltration, comprising administering a phenylbutyric acid or a pharmacologically acceptable salt thereof.
(8) The method according to (7), wherein the phenylbutyric acid is 4-phenyl-n-butyric acid or a pharmacologically acceptable salt thereof.
(9) A method of inhibiting a decrease of goblet cells, comprising administering a phenylbutyric acid or a pharmacologically acceptable salt thereof.
(10) The method according to (9), wherein the phenylbutyric acid is 4-phenyl-n-butyric acid or a pharmacologically acceptable salt thereof.
(11) A method of inhibiting a decrease in tear secretion, comprising administering a phenylbutyric acid or a pharmacologically acceptable salt thereof.
(12) The method according to (11), wherein the phenylbutyric acid is 4-phenyl-n-butyric acid or a pharmacologically acceptable salt thereof.

### Effects of the Invention

According to the present invention, a novel therapeutic or prophylactic agent for graft-versus-host disease, a novel agent for inhibiting fibrocyte infiltration, and a novel agent for inhibiting a decrease in tear secretion and a decrease of goblet cells can be provided.

### BRIEF DESCRIPTION OF THE DRAWINGS

FIG. 1A and FIG. 1D show that the levels of endoplasmic reticulum stress markers were increased in cGVHD affected organs. FIG. 1A shows the results from qPCR analysis of GRP78 in cGVHD affected organs and controls. FIG. 1B shows the results from immunoblot analysis of endoplasmic reticulum stress markers and related inflammation markers. FIG. 1C shows the results from quantitative analysis of the corresponding protein bands. FIG. 1D shows electron micrographs of epithelial cells in a cGVHD-affected lacrimal gland and a syngeneic control lacrimal gland thereof.

FIG. 2A and FIG. 2B show the %survival and change in weight for a PBA-treated mouse group and a vehicle-treated mouse group.

FIG. 3-1A shows the results from PBA inhibition of endoplasmic reticulum stress resulting from cGVHD. FIG. 3-1A shows the results from immunoblot analysis of the protein levels of endoplasmic reticulum stress markers and related inflammation markers in cGVHD target organs.

FIG. 3-2B shows the results from PBA inhibition of endoplasmic reticulum stress resulting from cGVHD. FIG. 3-2B shows the results from densitometric quantification of target proteins in PBA-treated organs and vehicle-treated organs.

FIG. 4-1A TO FIG. 4-1C show that cGVHD-induced systemic inflammation and fibrosis were reduced by allowing PBA to alleviate endoplasmic reticulum stress. FIG. 4-1A shows HE (hematoxylin and eosin) stained images of PBA-treated organs and vehicle-treated organs. FIG. 4-1B shows the results from immunostaining of CD45, a common leukocyte marker in PBA-treated tissues and vehicle-treated tissues. FIG. 4-1C shows the results from Mallory staining of PBA-treated organs and vehicle-treated organs.

FIG. 4-2D TO FIG. 4-2G show that cGVHD-induced systemic inflammation and fibrosis were reduced by allowing PBA to alleviate endoplasmic reticulum stress. FIG. 4-2D shows electron micrographs of a PBA-treated lacrimal gland and small intestine and a vehicle-treated lacrimal gland and small intestine. FIG. 4-2E shows the densities of CD45 positive cells in PBA-treated organs and vehicle-treated organs. FIG. 4-2F shows the results from PAS (Periodic acid-Schiff stain) staining of a PBA-treated small intestine and a PBA-treated eye; and a vehicle-treated small intestine and a vehicle-treated eye. FIG. 4-2G shows the densities of goblet cells in PBA treated small intestines, PBA treated eyes, vehicle treated small intestines, and vehicle treated eyes.

FIG. 5A to FIG. 5C show that markers for inflammation and fibrosis were each decreased by allowing PBA to alleviate endoplasmic reticulum stress. FIG. 5A shows the results from ELISA performed 28 days after bone marrow transplantation to measure inflammation markers MCP-1, TNF-α, and IFN-γ in blood sera collected from PBA-treated mice and vehicle-treated mice. FIG. 5B shows the results from immunoblot analysis of fibrosis marker CTGF. FIG. 5C shows the results from densitometric quantification of CTGF in each organ (PBA-treated organs and vehicle-treated organs).

FIG. 6A TO FIG. 6D shows that cGVHD-induced endoplasmic reticulum stress in lacrimal gland fibroblasts were alleviated by PBA, an agent for alleviating endoplasmic reticulum stress. FIG. 6A shows the results from immunoblot analysis of endoplasmic reticulum stress markers, activation markers, and fibrosis markers in mouse lacrimal gland fibroblasts. FIG. 6B shows the results of quantitative analysis of the corresponding protein bands (PBA-treated fibroblasts and vehicle-treated fibroblasts). FIG. 6C shows the results from ELISA measurements of the protein levels of MCP-1 produced in PBA-treated fibroblasts and vehicle-treated fibroblasts. FIG. 6D shows the results from qPCR analysis of IL-6 and TGF-β in PBA-treated fibroblasts and vehicle-treated fibroblasts.

FIG. 7A to FIG. 7E show the results from PBA alleviation of endoplasmic reticulum stress resulting from cGVHD in macrophages. FIG. 7A shows immunofluorescence images of a PBA-injected lacrimal gland and a vehicle-injected lacrimal gland. FIG. 7B shows the results from immunofluorescence of cultured splenic macrophages from a PBA-administered mouse and a vehicle-administered mouse. FIG. 7C shows the results from immunoblot analysis of endoplasmic reticulum stress markers in mouse splenic macrophages. FIG. 7D shows the results from quantitative analysis of the corresponding protein bands. FIG. 7E shows the results of qPCR analysis of M1 macrophage markers and M2 macrophage markers from spleens.

FIG. 8 shows a putative correlation between cGVHD and endoplasmic reticulum stress.

FIG. 9 shows the overview of the endoplasmic reticulum stress signaling pathways.

FIG. 10 also shows the overview of the endoplasmic reticulum stress signaling pathways.

### MODE FOR CARRYING OUT THE INVENTION

Specific embodiments of the present invention are described below in detail. The scope of the present invention, however, is not limited to these embodiments. Within the scope of the objects of the present invention, appropriate modification can be applied to the present invention. Some overlapping description is omitted in the following description, and such omission does not limit the scope of the present invention.

### <Therapeutic or Prophylactic Agent for Graft-Versus-Host Disease>

A therapeutic or prophylactic agent for graft-versus-host disease of the present invention contains a phenylbutyric acid or a pharmacologically acceptable salt thereof as an active ingredient.

### (Phenylbutyric Acid or Pharmacologically Acceptable Salt Thereof)

It is preferable that the phenylbutyric acid of the present invention be 4-phenyl-n-butyric acid (hereinafter may simply be referred to as “PBA.”) of the following formula.

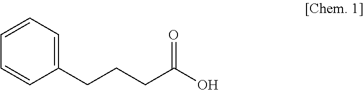 [SMILES: O=C(O)CCCc1ccccc1]

In the present invention, the term “fibrocyte infiltration” refers to infiltration of blood fibrocytes into tissue (for example, a lacrimal gland) and the term “inhibiting (or inhibition of) fibrocyte infiltration” refers to action that leads to inhibition of fibrocyte infiltration into tissue. Further, a person skilled in the art would recognize that the term “fibrocyte” would be used interchangeably with the term “fibroblast” (Disease Models & Mechanisms 2011 May; 4(3) 318-333 (in particular see page 325).

The phenylbutyric acid or a pharmacologically acceptable salt thereof of the present invention can be obtained by a known method. For example, the phenylbutyric acid or a pharmacologically acceptable salt thereof of the present invention can be obtained by the following methods.

The phenylbutyric acid of the present invention (for example, 4-Phenyl-n-butyric acid (CAS registry number: 1821-12-1)) can be obtained as a reagent or an industrial raw material, for example, or can be synthesized by a conventional procedure.

A pharmacologically acceptable salt of the phenylbutyric acid of the present invention is not limited provided that it can form a salt with a carboxy group. Specific examples of the pharmacologically acceptable salt include alkali metal salts, alkaline-earth metal salts, amine salts, and basic amino acid salts. Among these, a sodium salt, a potassium salt, a calcium salt, and a magnesium salt are more preferable.

The phenylbutyric acid or a pharmacologically acceptable salt thereof of the present invention may be in a form of a derivative thereof (so-called prodrug), such as an ester or an ether, that is readily hydrolyzed in a living organism (on the surface thereof) after administration. The phenylbutyric acid of the present invention may also be in a form of a structural isomer thereof, such as 3-phenyl-n-butyric acid (CAS registry number: 4593-90-2), 2-phenyl-n-butyric acid (CAS registry number: 90-27-7), 2-phenylisobutyric acid (CAS registry number: 826-55-1), or 3-phenylisobutyric acid (CAS registry number: 77-83-8).

### (Graft-Versus-Host Disease)

The graft-versus-host disease of the present invention is not particularly limited in type, and examples thereof include ones that show symptoms in organs having an exocrine gland, such as eyes, an oral cavity, liver, a digestive tract, skin, and lungs. Among these, ocular graft-versus-host disease is preferable to be treated or prevented with the therapeutic or prophylactic agent of the present invention.

Ocular graft-versus-host disease herein is not particularly limited, and examples thereof include dry eye, conjunctivitis sicca, corneal injury, or the like, and conjunctival fibrosis resulting from dry eye. Among these, dry eye is preferable as the ocular graft-versus-host disease to be treated or prevented because dry eye is particularly susceptible to the treatment or prevention.

Graft-versus-host disease in the oral cavity herein is not particularly limited, and examples thereof include oral dryness and trismus resulting from sclerema.

Graft-versus-host disease in the liver herein is not particularly limited, and examples thereof include acute hepatitis.

Graft-versus-host disease in the digestive tract herein is not particularly limited, and examples thereof include diarrhea, decreased appetite, vomiting, and obstruction and intestinal infarction (obstruction) in the upper gastrointestinal tract.

Graft-versus-host disease in skin herein is not particularly limited, and examples thereof include dry skin and scleroderma.

Graft-versus-host disease in a lung herein is not particularly limited, and examples thereof include interstitial pneumonia and obstructive pneumonia.

The organ to undergo transplantation and to have graft-versus-host disease is not particularly limited, and examples thereof include bone marrow and blood (blood transfusion). Reports suggest the following possibility: manifestation of graft-versus-host disease be caused by differentiation of bone marrow hematopoietic stem cells (especially ones after bone marrow transplantation) into fibrocytes (which are a cause of graft-versus-host disease) and infiltration of the fibrocytes into blood and then into exocrine gland tissue. The therapeutic or prophylactic agent of the present invention has excellent effect of inhibiting fibrocyte infiltration and thereby exhibits particularly excellent effect of treating or preventing such graft-versus-host disease that manifests after bone marrow transplantation. Therefore, it is preferable that the graft-versus-host disease to be treated or prevented with the therapeutic or prophylactic agent of the present invention be a graft-versus-host disease that manifests after bone marrow transplantation.

The graft-versus-host disease may be either chronic graft-versus-host disease (cGVHD) or acute graft-versus-host disease (acute GVHD: aGVHD). Typically, chronic graft-versus-host disease refers to a type of graft-versus-host disease that manifests a certain period of time after transplantation. According to traditional classification, chronic graft-versus-host disease refers to a type of graft-versus-host disease that manifests 100 days or later after transplantation, for example. Typically, acute graft-versus-host disease refers to a type of graft-versus-host disease that manifests within a certain period of time (shorter than the time for manifestation of chronic graft-versus-host disease) after transplantation. According to traditional classification, acute graft-versus-host disease refers to a type of graft-versus-host disease that manifests within 100 days after transplantation, for example. Currently, however, chronic and acute graft-versus-host diseases are distinguished from each other not with respect to the time period but with respect to signs typical of and characteristic to respective types. The therapeutic or prophylactic agent for graft-versus-host disease of the present invention can be particularly suitable for chronic graft-versus-host disease, probably because of the effect of the agent to inhibit fibrosis.

The therapeutic or prophylactic agent of the present invention can be used for all of mild, moderate, and severe graft-versus-host diseases, and can be particularly suitable for treating or preventing severe graft-versus-host disease because serious dry eye is accompanied by a noticeable level of fibrosis. Typically, severe graft-versus-host disease refers to ocular graft-versus-host disease with severe injury to lacrimal gland cells that secret tear, epithelial cells that secret corneal and conjunctival mucin, and meibomian gland cells.

As described above, the phenylbutyric acid or a pharmacologically acceptable salt thereof of the present invention can inhibit fibrocyte infiltration particularly effectively. Therefore, it is particularly preferable that the graft-versus-host disease of the present invention be one that is accompanied by fibrocyte infiltration.

### (Dosage Form)

The dosage form of the therapeutic or prophylactic agent of the present invention is not particularly limited and may be selected as appropriate depending on the organ with a symptom of graft-versus-host disease. For example, the therapeutic or prophylactic agent of the present invention can be in an ointment form, an injectable form for intravenous injection (including infusion), intramuscular injection, intraperitoneal injection, subcutaneous injection, or other types of injection, a suppository form, or a form for intratumoral direct administration. The injectable form of the agent may be contained in a single-dose ampule or in a multi-dose container. For treating or preventing ocular graft-versus-host disease, in particular, the therapeutic or prophylactic agent of the present invention is preferably in a form of eye drops, a subconjunctival injectable, or ointment.

Production of these various formulations may be conducted by a conventional procedure with appropriate use of additional components that are typically used for formulation purposes, such as an excipient, a filler, a binder, a wetting agent, a disintegrating agent, a lubricant, a pH adjusting agent, a surfactant, a dispersant, a buffer, a preservative, a dissolution promoter, an antiseptic, a flavoring agent, an anesthetic, a stabilizer, and a tonicity adjuster.

For formulation purposes, the therapeutic or prophylactic agent of the present invention may or may not contain a component other than the phenylbutyric acid or a pharmacologically acceptable salt thereof. Specific examples of the component that may be contained in the therapeutic or prophylactic agent of the present invention include methylcellulose.

### (Method of Administration)

The method of administering the therapeutic or prophylactic agent of the present invention is not particularly limited and may be selected as appropriate depending on the organ with a symptom. For example, administration thereof may be conducted by intravenous injection, intramuscular injection, intraperitoneal injection, subcutaneous injection, or the like. For ocular graft-versus-host disease, in particular, administration thereof may be conducted by ophthalmic administration or subconjunctival injection.

The method of administering the therapeutic or prophylactic agent of the present invention can be appropriately selected depending on the age and conditions of a patient. A dose of the therapeutic or prophylactic agent of the present invention may vary depending on the age, the route of administration, and the frequency of administration, and can be appropriately selected by a person skilled in the art. There is no particular limitation for the dose, but it is generally such that the amount of a phenylbutyric acid or a pharmaceutically acceptable salt thereof is in the magnitude of 0.1 μg to 10 mg per kg of weight per dose for oral or transvenous administration. In the case of an eye drop, one to several drops at one time may be administered once to several times daily, but the regimen may vary depending on symptoms. In the case of an ophthalmic ointment, an appropriate amount of the ointment may be applied once to several times daily.

### <Method of Screening>

The present invention subsumes a method of screening for a candidate substance for a therapeutic or prophylactic agent for graft-versus-host disease. The method comprises a step of administering test substances to non-human animal models of graft-versus-host disease, a step of measuring the extent of inhibition of fibrocyte infiltration or the extent of inhibition of endoplasmic reticulum stress in the animal models to which the test substances have been administered, and a step of selecting, from the test substances, a candidate substance for a therapeutic or prophylactic agent for graft-versus-host disease based on the measurement;

a method of screening, comprising a step of evaluating effects of the test substances on inhibiting infiltration of inflammatory cytokine-stimulated fibroblasts into resected or cultured lacrimal gland tissues; and the like.

### (Administration Step)

In the administration step of the present invention, test substances are administered to non-human animal models of graft-versus-host disease.

The test substances are not particularly limited and may be any substances regardless of whether they are natural or synthetic, organic or inorganic, or low-molecular or high-molecular.

The type of the non-human animal models of graft-versus-host disease is not particularly limited, and examples thereof include mammals such as mice, rats, dogs, cats, monkeys and apes, pigs, cows, sheep, and rabbits.

As an example of the animal models of graft-versus-host disease, a mouse model of graft-versus-host disease can be prepared by transplanting bone marrow derived from a male B10.D2 (H-2^{d}) mouse into a female BALB/c (H-2^{d}) mouse.

Suitably, excessive inflammation and fibrosis observed in the above model mouse with graft-versus-host disease are significantly similar to those observed in humans. Further, systemic inflammation and fibrosis are observed in the above model mouse (Yaguchi S, Ogawa Y, Shimmura S, et al. Angiotensin II Type 1 Receptor Antagonist Attenuates Lacrimal Gland, Lung, and Liver Fibrosis in a Murine Model of Chronic Graft-Versus-Host Disease. PLoS One. 2013; 8:1-11.; Ogawa Y, Morikawa S, Okano H, et al. MHC-compatible bone marrow stromal/stem cells trigger fibrosis by activating host T cells in a scleroderma mouse model. eLife. 2016; 5:e09394.).

The method of administration is not particularly limited and may be selected as appropriate, for example, depending on the target organ with a symptom. For example, administration may be conducted by intravenous injection, intramuscular injection, intraperitoneal injection, subcutaneous injection, or the like. In the case that the target organ with a symptom is an eye, administration may be conducted by subconjunctival injection or ophthalmic administration.

### (Measurement Step)

In the measurement step of the present invention, measurement is conducted regarding the extent of inhibition of fibrocyte infiltration in the animal models of graft-versus-host disease to which the test substances have been administered.

The method of measurement is not particularly limited and may be a conventionally known method. For example, the measurement of the extent of inhibition of fibrocyte infiltration or the extent of inhibition of endoplasmic reticulum stress may be conducted by resecting the target tissue (exocrine gland tissue such as lacrimal gland) from a non-human animal model of graft-versus-host disease after administration of a test substance, detecting a fibrocyte marker (such as CD45, type I collagen, or CXCR4) or an endoplasmic reticulum stress marker (for example, GRP78, CHOP, p-PERK, p-eIF2α, p-IRE1α, and the like) by a method such as immunostaining, and measuring the extent of fibrocyte infiltration (for example, the area of marker detection) or the extent of inhibition of endoplasmic reticulum stress. The results of the measurement can be used to select a candidate substance in the selection step, which is described below.

In the measurement step of the present invention, a factor other than the extent of inhibition of fibrocyte infiltration and the extent of inhibition of endoplasmic reticulum stress may or may not be measured as well. Examples of the factor that may be measured include the number of goblet cells in mucosa, the extent of inflammation in tissue, and the expression level of mRNA of a marker gene (for example, a marker gene for fibrosis (prefibrotic mediator connective tissue growth factor (CTGF)) and a marker gene for inflammation (for example, NF-κB, TXNIP, MCP-1, TNF-α, IFN-γ, and the like)). The results of this measurement may be combined with the results of the measurement of the extent of inhibition of fibrocyte infiltration and used in the selection step described below for selecting a candidate substance for a therapeutic or prophylactic agent for graft-versus-host disease from the test substances. It is noted that CTGF is known to serve as a fibrosis marker (Dziadzio M, Usinger W, Leask A, et al. N-terminal connective tissue growth factor is a marker of the fibrotic phenotype in scleroderma. QJM: An International Journal of Medicine. 2005; 98:485-492.).

### (Selection Step)

In the selection step, the results of the measurement above is used to select a candidate substance for a therapeutic or prophylactic agent for graft-versus-host disease from the test substances.

The method of selecting a candidate substance is not particularly limited, and may be any method conventionally adopted for selecting a candidate substance. For example, one of the test substances may be selected as a candidate substance when it is found that administration of this test substance has inhibited fibrocyte infiltration in tissue of a non-human animal model of graft-versus-host disease in consideration of the extent of fibrocyte infiltration measured before administration. Alternatively, one of the test substances may be selected as a candidate substance based on comparison between the results of administration of this test substance to a non-human animal model of graft-versus-host disease and the results previously obtained with respect to administration of another substance (such as a phenylbutyric acid or a pharmacologically acceptable salt thereof) (for example, a test substance may be selected as a candidate substance when the results of administration of this test substance to a non-human animal model of graft-versus-host disease are equivalent to the results of administration of a phenylbutyric acid or a pharmacologically acceptable salt thereof that is effective in treating graft-versus-host disease).

In the case that a factor other than the extent of inhibition of fibrocyte infiltration is also measured in the measurement step (for example, the number of goblet cells in mucosa, the extent of inflammation in tissue, and the expression level of mRNA of a marker gene), the results of this measurement may be combined with the results of measurement of the extent of inhibition of fibrocyte infiltration and used for selecting a candidate substance for a therapeutic or prophylactic agent for graft-versus-host disease from the test substances. For example, one of the test substances with a confirmed effect of inhibiting fibrocyte infiltration may be selected as a candidate substance for a therapeutic or prophylactic agent for graft-versus-host disease after the following procedures: after administration of this test substance to an animal model, measurement is conducted regarding one or two or more factors selected from the number of goblet cells in mucosa, the extent of inflammation in tissue, and the expression level of mRNA of a marker gene, and then it is confirmed that this test substance has effects regarding the measured factor or factors (that is, one or two or more effects among the effect of inhibiting a decrease in the number of goblet cells in mucosa, the effect of inhibiting inflammation in tissue, and the effect of decreasing or increasing the expression level of mRNA of a marker gene).

### (Other Steps)

The method of screening of the present invention may or may not comprise another step in addition to the steps described above. This another step that may be comprised in the method of screening of the present invention is not particularly limited, and examples thereof include a step of administering a test substance that has been selected as a candidate substance for a therapeutic or prophylactic agent for graft-versus-host disease to a non-human animal model of graft-versus-host disease and checking whether this test substance has an effect to treat or prevent graft-versus-host disease.

### <Agent for Inhibiting Fibrocyte Infiltration>

The present invention subsumes an agent for inhibiting fibrocyte infiltration. The agent for inhibiting fibrocyte infiltration contains a phenylbutyric acid or a pharmacologically acceptable salt thereof as an active ingredient.

The agent for inhibiting fibrocyte infiltration of the present invention may be equivalent to the therapeutic or prophylactic agent for graft-versus-host disease described above (in terms of the phenylbutyric acid or a pharmacologically acceptable salt thereof, the dosage form, the method of administration, the components, and the like).

### <Agent for Inhibiting Decrease of Goblet Cells>

The present invention comprises an agent for inhibiting a decrease of goblet cells. The agent for inhibiting a decrease of goblet cells contains a phenylbutyric acid or a pharmacologically acceptable salt thereof as an active ingredient. It is preferable that the goblet cells be conjunctival goblet cells.

The agent for inhibiting a decrease of goblet cells of the present invention may be equivalent to the therapeutic or prophylactic agent for graft-versus-host disease described above (in terms of the phenylbutyric acid or a pharmacologically acceptable salt thereof, the dosage form, the method of administration, the components, and the like).

### <Agent for Inhibiting Decrease in Tear Secretion>

The present invention comprises an agent for inhibiting a decrease in tear secretion. The agent for inhibiting a decrease in tear secretion contains a phenylbutyric acid or a pharmacologically acceptable salt thereof as an active ingredient.

In the present invention, the term “a decrease in tear secretion” refers to decreased tear secretion that manifests as a symptom of graft-versus-host disease described above (for example, dry eye).

The agent for inhibiting a decrease in tear secretion of the present invention may be equivalent to the therapeutic or prophylactic agent for graft-versus-host disease described above (in terms of the phenylbutyric acid or a pharmacologically acceptable salt thereof, the dosage form, the method of administration, the components, and the like).

### <Method of Treating or Preventing Graft-Versus-Host Disease>

The present invention comprises a method of treating or preventing graft-versus-host disease. This method comprises administering a phenylbutyric acid or a pharmacologically acceptable salt thereof to a patient in need of treatment or prevention of graft-versus-host disease. In this method of the present invention, the following factors may be equivalent to the corresponding factors regarding the therapeutic or prophylactic agent for graft-versus-host disease described above: factors such as the phenylbutyric acid or a pharmacologically acceptable salt thereof, the dosage form, the method of administration, and the components.

### <Method of Inhibiting Fibrocyte Infiltration>

The present invention comprises a method of inhibiting fibrocyte infiltration. This method comprises administering a phenylbutyric acid or a pharmacologically acceptable salt thereof to a patient in need of inhibition of fibrocyte infiltration. In this method of the present invention, the following factors may be equivalent to the corresponding factors regarding the agent for inhibiting fibrocyte infiltration: factors such as the phenylbutyric acid or a pharmacologically acceptable salt thereof, the dosage form, the method of administration, and the components.

### <Method of Inhibiting Decrease of Goblet Cells>

The present invention comprises a method of inhibiting a decrease of goblet cells. This method comprises administering a phenylbutyric acid or a pharmacologically acceptable salt thereof to a patient in need of inhibition of a decrease of goblet cells. In this method of the present invention, the following factors may be equivalent to the corresponding factors regarding the agent for inhibiting a decrease of goblet cells: factors such as the phenylbutyric acid or a pharmacologically acceptable salt thereof, the dosage form, the method of administration, and the components.

### <Method of Inhibiting Decrease in Tear Secretion>

The present invention comprises a method of inhibiting a decrease in tear secretion. This method comprises administering a phenylbutyric acid or a pharmacologically acceptable salt thereof to a patient in need of inhibition of a decrease in tear secretion. In this method of the present invention, the following factors may be equivalent to the corresponding factors regarding the agent for inhibiting a decrease in tear secretion: factors such as the phenylbutyric acid or a pharmacologically acceptable salt thereof, the dosage form, the method of administration, and the components.

### EXAMPLES

Below, the present invention will be described with reference to Examples. However, the present invention shall not be limited to the descriptions of the following Examples.

### <Method>

### (Bone Marrow Transplantation)

B10.D2 mice and BALB/c mice of 8 weeks old were purchased from Sankyo Research Laboratories (Tokyo, Japan).

Bone marrow transplantation was performed on model mice of cGVHD (Zhang Y, McCormick L L, Desai S R, Wu C, Gilliam A C, Murine Sclerodermatous Graft-Versus-Host Disease, a Model for Human Scleroderma: Cutaneous Cytokines, Chemokines, and Immune Cell Activation. The Journal of Immunology, 2002; 168: 3088-3098). When a donor was a B10.D2 mouse and a recipient was a BALB/c mouse, bone marrow transplantation was allogeneic, producing a model mouse of cGVHD. In contrast, bone marrow transplantation from a BALB/c mouse to another BALB/c mouse was syngeneic, and thus cGVHD did not manifest in a transplant recipient. Recipient mice in which cGVHD did not manifest were used as controls. Recipients were irradiated at 700 cGy before bone marrow transplantation to perform lethal irradiation with a Gammacel 137 Cs source (Hitachi Medical Corp.). The recipient mice were each administered via tail vein with a suspension containing 1×10⁶ donor bone marrow cells and 2×10⁶ donor spleen cells.

The donor cells were suspended in RPMI 1640 (Life Technologies Japan Ltd.).

### (PBA Treatment of Allogeneic Bone Marrow Transplantation Recipient Mice)

Bone marrow transplantation was performed as described in the above section “Bone marrow transplantation,” and the resulting allogeneic bone marrow transplantation recipient mice were divided into two groups. One group was treated with intraperitoneal injection of PBA (10 mg/kg) (Aldrich) while the other was treated with intraperitoneal injection of a solvent vehicle⋅phosphate buffered saline (PBS) (pH 7.4). The allogeneic bone marrow transplantation recipients received once-daily administration of PBA or the solvent vehicle from Day 10 to Day 27 after bone marrow transplantation. They were sacrificed on Day 28 after bone marrow transplantation.

cGVHD-affected organs (exorbital lacrimal gland, proximal portion of the small intestine, dorsal skin, liver, salivary gland, lung, large intestine, and eyes) were analyzed herein.

### (Histological Analysis and Immunohistochemistry)

The exorbital lacrimal gland, the proximal part of the small intestine, dorsal skin, the liver, the salivary gland, the lung, the large intestine, and the eyes were collected from transplant recipients at 3 or 4 weeks after bone marrow transplantation. Subsequently, these samples were fixed with 10% neutral buffered formalin, and then each embedded in paraffin. The resulting paraffin blocks were each cut into 7-μm thick sections, and then stained with
(1) hematoxylin and eosin;
(2) Malory trichrome staining (Hopwood J, Fixation and fixtative. In: Bancroft J D, Stevens A, eds., Theory and Practice of Histological Techniques, 4th ed, Edinburgh: Churchill-Livingstone, 1996:23-46; Anderson G, Gordon K, Tissue processing, microtomy and paraffin sections. In: Bancroft J D, Stevens A, eds. T. Theory and Practice of Histological Techniques, 4th ed, Edinburgh: Churchill-Livingstone, 1996:47-68); and
(3) antibody.

For immunohistochemical assay, paraffin was first removed, and antigens were then collected by either one of the following two antigen collecting methods.
(A) For staining sections with CD45 antibody (30-F11, BD Pharmingen), the sections were immersed in an antigen retrieval solution (Target Retrieval Solution; Dako), and then boiled for 10 minutes in a microwave oven.
(B) For multistaining with CD68 (FA-11, Abd Serotec) and CHOP (F-168, Santa Cruz Biotechnology, Inc.), sections were washed with an antigen retrieval solution (HistoVT One; Nacalai Tesque, Inc.), and then heated in a water bath at 90° C. for 40 minutes. Next, the sections were blocked with 10% normal goat blood serum, and antigens in the tissue sections were then allowed to react with the primary antibodies at 4° C. overnight. Subsequently, the sections were treated with fluorophore-labelled secondary antibodies at room temperature (for example, 1 to 30° C.) for 45 minutes, and then mounted with an anti-fading mounting medium (Fluorescent Mounting Medium; Dako).

Fluorescence images were captured under an LSM confocal microscope (Carl Zeiss). For counting CD45⁺ cells, 5 randomly selected regions per section were photographed at a magnification of ×200, and the number of CD45⁺ cells in each image was then determined. As secondary antibodies, used were goat anti-mouse IgG (H+L) secondary antibody conjugated with Alexa Fluor 488 (Molecular Probes) and goat anti-rat IgG (H+L) secondary antibody conjugated with Alexa Fluor 568 (Molecular Probes). For isotype controls, rat IgG2b, κ (eB149/10H5, eBioscience, Inc.), rat IgG2a (54447, R&D Systems, Inc.), and rabbit IgG (Cell Signaling Technology, Inc.) were used for CD45, CD68, and CHOP, respectively.

### (Culture of Mouse Lacrimal Gland-Derived Fibroblasts)

Fibroblasts from a mouse lacrimal gland were cultured by an established method reported by Yaguchi et al. (Yaguchi S, Ogawa Y, Shimmura S, et al., Presence and Physiologic Function of the Renin—Angiotensin System in Mouse Lacrimal Gland. Investigative Ophthalmology & Visual Science, 2012; 53: 5416-5425). A mouse lacrimal gland was collected and cut into small pieces. The tissue pieces were incubated at 37° C. in DMEM (Life Technologies Japan Ltd.) containing 5% of fetal bovine serum (FBS) Sigma), and 5% of an antibiotic. The antibiotic included a 1:1 mixture of streptomycin sulfate (Meiji Seika Pharma Co., Ltd.) and benzylpenicillin potassium (Meiji Seika Kaisha, Ltd.). The small pieces were used to grow fibroblasts starting from 3 to 4 days later. Fibroblasts were cultured in DMEM containing 5% of FBS and 5% of the antibiotic, and were used in experiments after 3 to 5 passages. Trypsin (Becton Dickinson) was used to detach fibroblasts from the culture dish.

### (Culture of Splenic Macrophages)

Mouse splenic macrophages were cultured according to the Alatery and Besta method (Alatery A, Basta S, An efficient culture method for generating large quantities of mature mouse splenic macrophages. J Immunol Methods, 2008; 338: 47-57). Splenic cells suspended in RPMI containing 5% FBS were plated on a culture dish, and allowed to stand at 37° C. overnight. Subsequently, floating cells were removed. The desired cells adhering on the dish were cultured for 7 days in RPMI containing recombinant mouse M-CSF (5 ng/mL) (Peprotech Inc.), 5% of FBS, and 5% of the antibiotic. Induced macrophages were then detached from the culture dish by using accutase (Thermo Fisher Scientific), and used for experiments.

### (Immunostaining of Cultured Macrophages)

The induced macrophages were then transferred to an 8-well chamber slide (Fibronectin Culture Slide; Corning), and fixed with 10% neutral buffered formalin. Subsequently, they were blocked with methanol containing 0.3% hydrogen peroxide for 30 minutes at room temperature, and incubated with primary antibodies CD68 (AbD Serotec) and CHOP (Santa Cruz) overnight at 4° C. Then, the sections were treated with goat anti-mouse IgG (H+L) secondary antibody conjugated with Alexa Fluor 568 (Molecular Probes) and 4′,6-diamidino-2-phenylindole (DAPI) (Life Technologies) at room temperature for 45 minutes, and then mounted with an anti-fading mounting medium (Dako). Fluorescence images were captured under an LSM confocal microscope (Carl Zeiss).

### (Electron Microscopy)

Transmission electron microscopy was performed in accordance with a standard protocol. Tissues were collected from a mouse lacrimal gland and small intestine, and immediately fixed with 2.5% glutaraldehyde in 0.1 M phosphate buffer (pH 7.4) at 4° C. for 4 hours, and then washed 3 times with 0.1 M phosphate buffer. Subsequently, the samples were fixed again with 2% osmium tetroxide, and dehydrated with a gradient series of ethanol and 100% propylene oxide, and then embedded in epoxy resin. Sections each having a thickness of 1 μm were prepared from the treated tissues, and then stained with methylene blue. A thick section was observed under a microscope to find a portion suitable for preparing an ultrathin section. The resulting section was placed on a mesh grid, stained with uranyl acetate and lead citrate, and examined under an electron microscope (1230 EXIT; JOEL). All electron micrographs were captured with a bioscan camera (GATAN Bioscan Camera Model 792).

### (Quantitative Polymerase Chain Reaction)

The total RNA was extracted from the exorbital lacrimal gland, the proximal portion of the small intestine, the dorsal skin, the liver, the cultured fibroblasts, and the cultured macrophages using a miRNeasy mini kit (Qiagen), and the corresponding complementary DNAs were synthesized using a Rever Tra Ace qPCR RT kit (Toyobo Co., Ltd.). Primers used for analyzing the mRNA expression of the following genes with the TaqMan real-time polymerase chain reaction (PCR) were purchased from Applied Biosystems: housekeeping gene glyceraldehyde 3-phosphate (GAPDH), glucose-regulated protein 78 (GRP78), Interleukin-1β (IL-1β), IL-6, IL-10, macrophage chemoattractant protein-1 (MCP-1), and transcription growth factor-β (TGF). A Step One Plus system (Applied Biosystems) was used to perform quantitative real time PCR, and the resulting data was analyzed by the 2^{-ΔΔCT} method. GAPDH was used as an internal standard for measuring the expression of mRNA.

### (Immunoblotting Analysis)

Tissues of interest were each placed into an Eppendorf tube, and a pre-cooled RIPA buffer solution was then added to the tube. Subsequently, the tissues were each homogenized with an electric homogenizer. Samples were each allowed to stand on ice for 1 hour, and then centrifuged at 15000 rpm for 5 minutes at 4° C. Then, the supernatants were each collected in a fresh tube on ice, and used as a cell lysate. The cell lysates each received the same amount of a 5×Laemmli buffer solution, and each was heated at 100° C. for 5 minutes to denature proteins.

An aliquot of each sample having the same amount of proteins was loaded into a well of an SDS-PAGE gel, and then dissolved. Proteins were transferred from the gel to a membrane at 15 V for 20 minutes. The membrane was blocked at room temperature for 1 hour with 5% skim milk or 5% BSA (bovine serum albumin) in 1×TBST (a mixture of Tris-buffered saline and Tween 20). Subsequently, the membrane was incubated with a primary antibody at 4° C. overnight. The above primary antibody was diluted 1000 times with 5% skim milk or 5% BSA in 1×TBST. After incubation with the primary antibody, the membrane was washed with 1×TBST (3×10 minutes), and subjected to a secondary antibody at room temperature for 1 hour, and then washed with 1×TBST (3×10 minutes) and 1×TBS (2×10 minutes).

Proteins of interest were visualized by either one of the following two methods. (1) A target protein was colorimetrically detected using a BCIP/NBT substrate (Promega). (2) A signal was developed with an enhanced chemiluminescence (ECL) detection reagent (GE Healthcare), and a target protein was then visualized with an LAS4000 mini chemiluminescence imaging system (FUJIFILM Corporation/GE Healthcare).

The intensity of the resulting protein band was analyzed using image processing software ImageJ. Primary antibodies used in the experiments were GRP78 (Abcam), phospho-PERK (Thr980; Cell Signaling Technology Inc.), PERK (C33E10; Cell Signaling Technology Inc.), phosphor-IRE1α (Thermo Fisher Sceientific), IRE1α (14C10; Cell Signaling Technology Inc.), phosphor-eIF2α (119A11; Cell Signaling Technology Inc.), eIF2α (Cell Signaling Technology Inc.), CHOP (9C8; Thermo Fisher Scientific), TXNIP (D5F3E, Cell Signaling Technology Inc.), NF-κB (Abcam), HSP47 (SPA-470; Stress-Gen Biotechnologies Corp.), CTGF (Abcam), and β-actin (AC-15; Abcam).

With regard to the secondary antibody, (1) either AP-conjugated anti-mouse IgG antibody (Promega) or AP-conjugated anti-rabbit IgG antibody (Promega) was used when protein bands were chromogenically visualized; (2) HRP-conjugated anti-mouse antibody (Thermo Fisher Scientific) or HRP-conjugated anti-rabbit antibody (Thermo Fisher Scientific) was used when target proteins were detected by ECL.

### (Enzyme-Linked Immunosorbent Assay (ELISA))

Blood samples were withdrawn from PBA-treated mice and vehicle-treated mice, and centrifuged at 4000 rpm for 10 minutes. The levels of MCP-1, tumor necrosis factor-α (TNF-α), and interferon-γ (IFN-γ) in the resulting blood sera were measured using ELISA kits (Becton Dickinson). These assays were performed according to the protocol recommended by manufacturer Becton Dickinson.

### (Statistical Analysis)

Statistical significance was determined by the Mann-Whitney U test (which tests difference between two groups that have no correspondence). Difference is considered as significant in the case of P<0.05. The data obtained is shown as the mean±SD (standard deviation).

### <Results>

[Increased Levels of Endoplasmic Reticulum Stress Markers in cGVHD-Affected Organs]

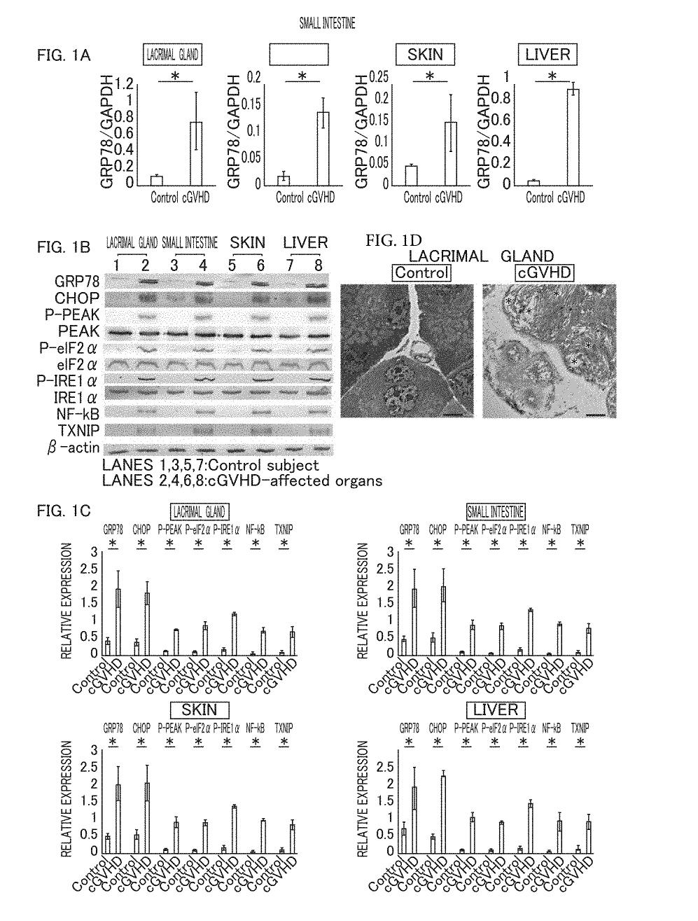

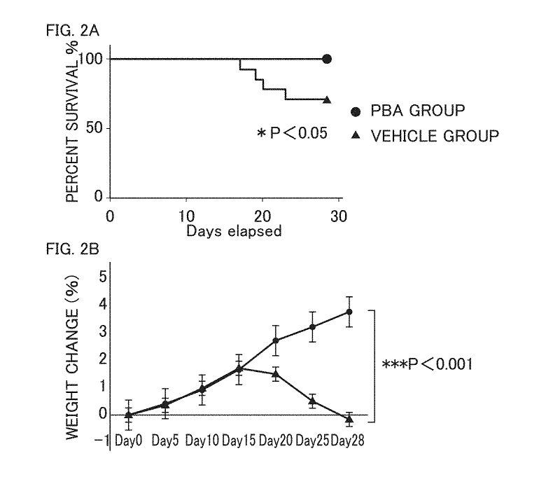

Endoplasmic reticulum stress markers were measured to investigate whether endoplasmic reticulum stress increased or not in cGVHD-affected organs. First, the levels of GRP78 in cGVHD-affected organs and controls were measured by qPCR analysis. Results are shown in FIG. 1A. In FIG. 1A, the data is shown as the mean±SD (control: n=5, cGVHD: n=4 to 5, and *P<0.05).

Endoplasmic reticulum stress markers and related inflammation markers were subjected to immunoblot analysis. Results are shown in FIG. 1B. In FIG. 1B, Lanes 1, 3, 5, and 7 correspond to syngeneic controls; and Lanes 2, 4, 6, and 8 correspond to cGVHD-affected organs.

Protein bands were each subjected to the corresponding quantitative analysis. Results are shown in FIG. 1C. In FIG. 1C, the data is shown as the mean±SD, control: n=4, cGVHD: n=4, and *P<0.05.

As clearly understood from the results shown in FIG. 1A to FIG. 1C, the real-time quantitative PCR (qPCR) and immunoblot analysis show the following:
(1) endoplasmic reticulum stress markers GRP78, CHOP, p-PERK, p-eIF2α, and p-IRE1α were increased in cGVHD-affected mouse organs as compared with the controls;
(2) in response to this, two inflammation markers NF-κB and TXNIP were activated and/or increased in cGVHD-affected organs.

FIG. 1D shows electron micrographs of epithelial cells in a cGVHD-affected lacrimal gland and a syngeneic control lacrimal gland. The images were captured at a magnification of ×2000. The scale bars indicate 5 μm. In the image of the cGVHD-affected lacrimal gland epithelium, the symbol “*” indicates a portion in which the endoplasmic reticulum was hypertrophied due to accumulation of proteins. As clearly seen in the electron micrographs shown in FIG. 1D, the endoplasmic reticulum underwent hypertrophy due to accumulation of misfolded/unfolded proteins in the cGVHD-affected lacrimal gland epithelium (indicated by * in FIG. 1D). In contrast, the endoplasmic reticulum in the control appeared to be unaffected. These findings showed that endoplasmic reticulum stress was increased in the cGVHD-affected organ.

### [Inactivation of Endoplasmic Reticulum Stress-Induced Inflammation Pathways by PBA]

Next, treatment of cGVHD by allowing PBA to reduce endoplasmic reticulum stress was tested. As described in the above section “Method,” mice that received allogeneic bone marrow transplantation were treated with PBA or a solvent vehicle. FIG. 2A to FIG. 2B show % survival (FIG. 2A) and change in weight (FIG. 2B) for a PBA-treated mouse group and a vehicle-treated mouse group. In FIG. 2A, the values are shown as the mean±SE (standard error) (n=14, and *P<0.05). In FIG. 2B, the values are shown as the mean±SE (n=10, and ***P<0.001). As clearly seen in FIG. 2A, the PBA-treated mouse group showed a higher survival percentage than the vehicle-treated mouse group. As clearly seen in FIG. 2B, the vehicle-treated mouse group showed larger weight loss as compared with the PBA-treated mouse group. Further, some mice in the vehicle-treated mouse group showed (1) hair loss and/or (2) lying-down.

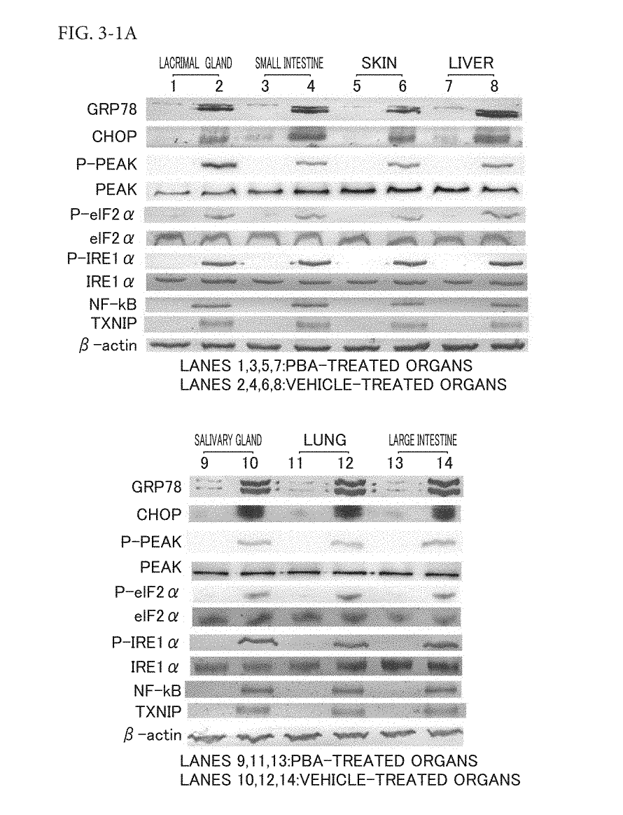

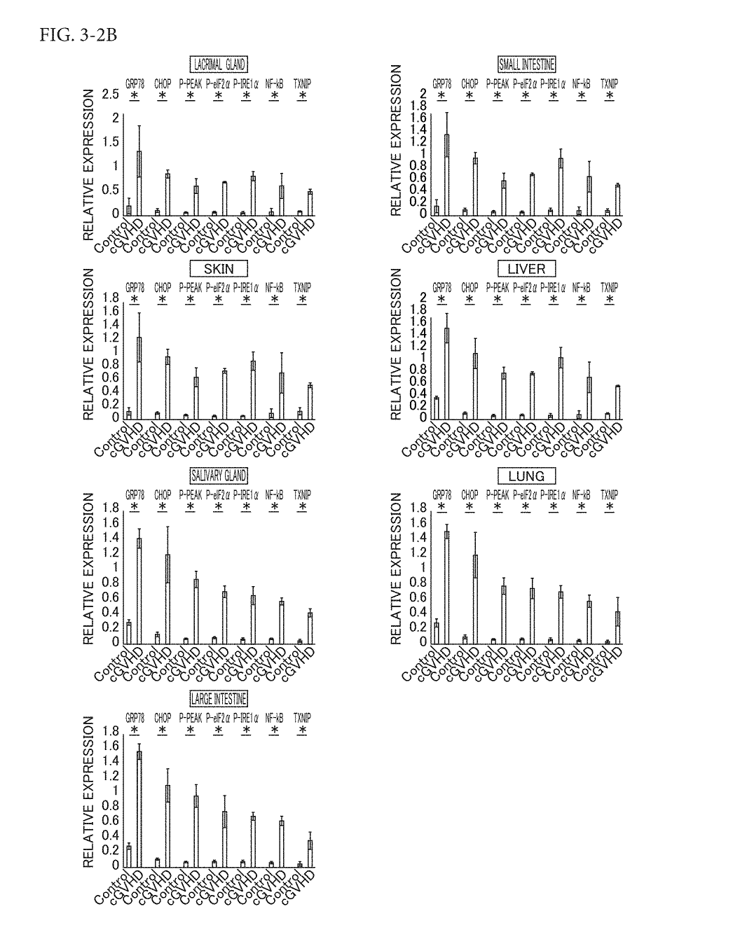

Next, immunoblot analysis was performed to test the protein levels of endoplasmic reticulum stress markers and related inflammation markers in cGVHD target organs. Results are shown in FIG. 3-1A. In FIG. 3-1A, Lanes 1, 3, 5, 7, 9, 11, and 13 correspond to PBA-treated organs; and Lanes 2, 4, 6, 8, 10, 12, and 14 correspond to vehicle-treated organs.

Subsequently, target proteins in the PBA-treated organs and the vehicle-treated organs were each densitometrically quantified. Results are shown in FIG. 3-1B. In FIG. 3-1B, the data is shown as the mean±SD (PBA: n=4, vehicle: n=4, and *P<0.05).

As clearly understood from the results shown in FIG. 3-1A and FIG. 3-1B, the immunoblot analysis indicates that the organs obtained from the PBA-treated mice had lower protein levels of GRP78, CHOP, p-PERK, p-eIF2α, and p-IRE1α as compared with those obtained from the vehicle-treated mice. As a result of this, related proinflammatory molecules NF-κB and TXNIP were found to be inhibited in the PBA-treated organs as compared with in the vehicle-treated organs, as shown in FIG. 3-1A and FIG. 3-1B. That is, the results show that endoplasmic reticulum stress resulting from cGVHD in mouse organs can be alleviated by PBA, which in turn can lead to significant inhibition of endoplasmic reticulum stress-related proinflammatory molecules NF-κB and TXNIP. Meanwhile, epithelial-mesenchymal transition is known to be related to fibrosis observed in human eyeball cGVHD while TXNIP is known to be related to epithelial-mesenchymal transformation (Ogawa Y, Shimmura S, Kawakita T, Yoshida S, Kawakami Y, Tsubota K, Epithelial Mesenchymal Transition in Human Ocular Chronic Graft-Versus-Host Disease. The American Journal of Pathology, 2009; 175: 2372-2381; Wei J, Shi Y, Hou Y, et al., Knockdown of thioredoxin-interacting protein ameliorates high glucose-induced epithelial to mesenchymal transition in renal tubular epithelial cells. Cell Signal, 2013; 25: 2788-2796). This suggests that PBA can alleviate cGVHD-induced systemic fibrosis by decreasing the expression of TXNIP to eliminate epithelial-mesenchymal transformation.

[Histological Observation of cGVHD Target Organs]

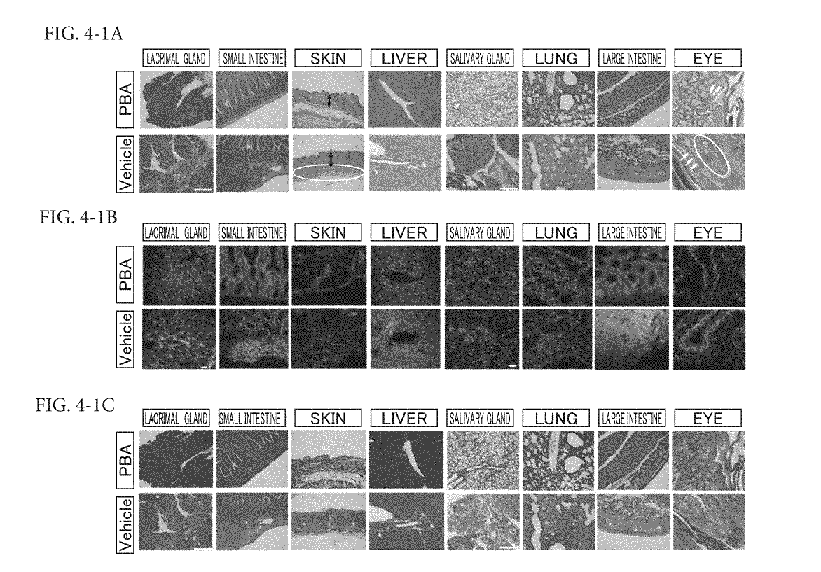

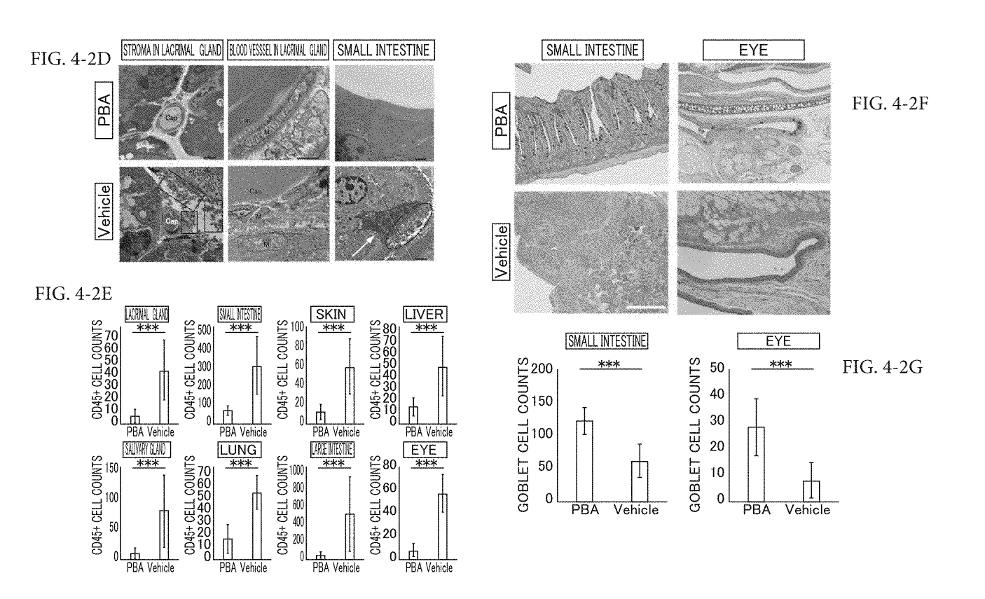

Alleviation of endoplasmic reticulum stress by PBA reduced cGVHD-induced systemic inflammation and fibrosis. Results are shown in FIG. 4-1A to FIG. 4-2G. FIG. 4-1A shows HE (hematoxylin and eosin) stained images of PBA-treated organs, and HE-stained images of solvent vehicle-treated organs. In FIG. 4-1A, images were each captured at a magnification of ×200, and shown with a scale bar of 20 μm. Portions with serious inflammation are indicated by the symbol “*”. In the image of the vehicle-treated skin, a portion where fat tissues were lost is enclosed by an ellipse. Further, the thicknesses of the PBA-treated skin and vehicle-treated skin are indicated by arrows. In the image of the vehicle-treated eye, a portion in which the meibomian gland was shrunk and contracted is marked with an ellipse, and the conjunctiva epithelium is indicated by an arrow.

FIG. 4-1B shows the results from immunostaining of CD45, a common leukocyte marker in PBA-treated tissues and vehicle-treated tissues. In FIG. 4-1B, images were each captured at a magnification of ×200, and shown with a scale bar of 200 μm. The cytosols and nuclei in cells were stained with red and blue, respectively.

FIG. 4-1C shows the results from Mallory staining of PBA-treated organs and vehicle-treated organs. In FIG. 4-1C, images were each captured at a magnification of ×200. The scale bars indicate 200 μm. An abnormal fibrosis region is indicated by the symbol “*”.

FIG. 4-2D shows electron micrographs of a PBA-treated lacrimal gland and small intestine and a vehicle-treated lacrimal gland and small intestine. In FIG. 4-2D, the images of stromata in the lacrimal glands (left) and epithelial cells in the small intestines (right) were captured at a magnification of ×2000. The scale bars indicate 5 μm. The images of the blood vessels of the lacrimal glands were captured at a magnification of ×15000. The scale bars indicate 500 nm. In FIG. 4-2D, “Cap” indicates a capillary vessel while “M” indicates a mitochondrion. In the image of the vehicle-treated lacrimal gland, a portion where an endoplasmic reticulum underwent hypertrophy due to accumulation of proteins is indicated by the symbol “*”, and cell fragments are marked by rectangles. In the image of the vehicle-treated small intestine, portions where the microvillus was destroyed are enclosed by an ellipse, a damaged tissue is indicated by an arrow.

FIG. 4-2E shows the densities of CD45 positive cells in PBA-treated organs and vehicle-treated organs. In FIG. 4-2E, the data is shown as the mean±SD (PBA: n=3, vehicle: n=3, *P<0.05, **P<0.01, and ***P<0.001).

FIG. 4-2F shows the results from PAS (Periodic acid-Schiff stain) staining of a PBA-treated small intestine and a PBA-treated eye; and a vehicle-treated small intestine and a vehicle-treated eye. Images were captured at a magnification of ×200. The scale bars indicate 200 μm. Goblet cells were stained as purple dots.

FIG. 4-2G shows the densities of goblet cells in PBA treated-small intestines, PBA treated eyes, vehicle-treated small intestines, and vehicle-treated eyes. In FIG. 4-2G, the numerical values are shown as the mean±SD (PBA: n=3 (small intestine), vehicle: n=3 (small intestine), PBA: n=5 (eye), vehicle: n=5 (eye), ***P<0.001).

The HE (hematoxylin and eosin) staining and Malory (trichrome) staining of cGVHD-affected organs shown in FIG. 4-1A and FIG. 4-1C show that reduction of endoplasmic reticulum stress by means of PBA can be an effective method of treating cGVHD. The HE images shown in FIG. 4-1A indicate that infiltration of inflammatory cells was inhibited in the PBA-treated organs as compared with the vehicle-treated organs. In particular, the images show that (1) the intestinal villi of the PBA-treated small and large intestines were not impaired in contrast with those of the solvent vehicle-treated small and large intestines;
(2) the vehicle-treated skin became thickened, lost fat tissues, and increased the density of collagen bundles in contrast with the PBA-treated skin;
(3) the meibomian gland of the vehicle-treated eye was decreased and contracted as compared with the PBA-treated meibomian gland; and
(4) thinning and damage were observed in the vehicle-treated conjunctiva epithelium in contrast with the PBA-treated conjunctiva epithelium, suggesting that symblepharon can be prevented by systemic injection of PBA.

As understood from the immunostaining and subsequent counting of CD45⁺ cells shown in FIG. 4-1C and FIG. 4-2E, the numbers of inflammatory cells in the PBA-treated organs were considerably smaller than those in the vehicle-treated organs. These findings also indicate that migration and growth of immune cells in the PBA-treated organs were inhibited. Meanwhile, systemic fibrosis represents one of the most serious problems in cGVHD (Ogawa Y, Morikawa S, Okano H, et al., MHC-compatible bone marrow stromal/stem cells trigger fibrosis by activating host T cells in a scleroderma mouse model. eLife, 2016; 5: e09394).

The results from the Mallory staining shown in FIG. 4-1C reveal that cGVHD-induced systemic fibrosis was substantially inhibited by the PBA treatment as compared with the vehicle-treated organs. The results from the electron micrographs of the lacrimal glands shown in FIG. 4-2D indicate that in the solvent vehicle-treated lacrimal gland, (1) the endoplasmic reticula in epithelial cells and endothelial cells undergo hypertrophy due to accumulation of unfolded/misfolded proteins;
(2) a large amount of cell fragments is present in the stromata of the endoplasmic reticula; and
(3) the mitochondria in the intravascular epithelial cells and endothelial cells are damaged. These results suggest that PBA protects blood vessels from endoplasmic reticulum stress resulting from cGVHD, thereby inhibiting migration of immune cells into tissues.

Further, the results from the electron micrograph analysis of the small intestine shown in FIG. 4-2D indicate that in the solvent vehicle-treated small intestine, the small villi thereof were destroyed, and adjacent tissues were damaged. These histological characteristics were not observed in the PBA-treated lacrimal gland and the PBA-treated small intestine.

Further, the results from the PAS (Periodic acid-Schiff stain) staining shown in FIG. 4-2F and FIG. 4-2G indicate that (1) the PBA-treated small intestine and the PBA-treated conjunctiva epithelium had more goblet cells than the vehicle-injected counterparts; and (2) the structures of the intestinal and conjunctival mucous membranes remained intact after systemic injection of PBA. As understood from these histological observations, PBA can alleviate systemic inflammation and fibrosis induced by cGVHD-related endoplasmic reticulum stress.

### [Reduction of Inflammatory and Prefibrotic Mediators by PBA]

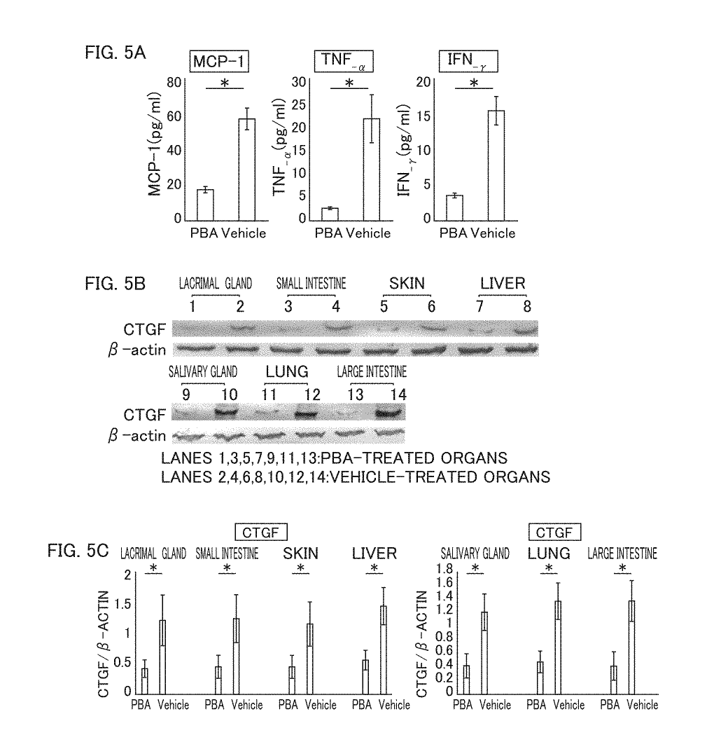

In order to closely investigate whether PBA can alleviate systemic inflammation resulting from cGVHD or not, ELISA was performed to measure the levels of inflammation markers MCP-1, TNF-α, and IFN-γ in blood sera collected from PBA-treated mice and vehicle-treated mice. FIG. 5A shows the results from ELISA performed 28 days after bone marrow transplantation to measure inflammation markers MCP-1, TNF-α, and IFN-γ in blood sera collected from PBA-treated mice and vehicle-treated mice. In FIG. 5A, the data is shown as the mean±SD (PBA: n=4, vehicle: n=4, and *P<0.05). As shown in FIG. 5A, PBA-treated mice had lower protein levels of inflammation markers MCP-1, TNF-α, and IFN-γ in blood sera as compared with vehicle-treated mice.

Alleviation of cGVHD-induced systemic fibrosis was further studied in more detail. FIG. 5B shows the results from immunoblot analysis of prefibrotic mediator connective tissue growth factor (CTGF) as a fibrosis marker. In FIG. 5B, Lanes 1, 3, 5, and 7 correspond to PBA-treated organs; and Lanes 2, 4, 6, and 8 correspond to vehicle-treated organs.

FIG. 5C shows the results from densitometric quantification of CTGF in each organ (PBA-treated organs and vehicle-treated organs). The data is shown as the mean±SD (PBA: n=4, vehicle: n=4, and *P<0.05).

As shown in FIG. 5B and FIG. 5C, the immunoblot analysis indicated that CTGF was overexpressed in the vehicle-treated organs, but not in the PBA-treated organs. This demonstrates that that PBA can inhibit cGVHD-induced overexpression of CTGF. The data presented in the above indicates that PBA can inhibit cGVHD-induced endoplasmic reticulum stress, leading to alleviation of systemic inflammation and fibrosis, and thus can alleviate cGVHD-induced physical disorders.

[Correlation between Endoplasmic Reticulum Stress and Fibroblast Dysfunction]

Systemic fibrosis resulting from cGVHD is a serious problem, and may cause multiple organ failure (Ogawa Y, Morikawa S, Okano H, et al., MHC-compatible bone marrow stromal/stem cells trigger fibrosis by activating host T cells in a scleroderma mouse model. eLife, 2016; 5: e09394). Although the mechanism of cGVHD-induced fibrosis remains to be elucidated, fibroblasts may be related to the development of fibrosis (Ogawa Y, Razzaque M S, Kameyama K, et al., Role of Heat Shock Protein 47, a Collagen-Binding Chaperone, in Lacrimal Gland Pathology in Patients with cGVHD. Investigative Ophthalmology & Visual Science, 2007; 48: 1079-1086). Several biological signals can stimulate fibroblasts, and play an essential role in several biological processes such as wound healing. However, activation of fibroblasts in an uncontrolled manner may result in formation of abnormal collagen bundles. This may induce serious fibrosis although little is known for the cause of this abnormality (Darby I A, Hewitson T D, Fibroblast Differentiation in Wound Healing and Fibrosis. International of Review of Cytology, 2007; 257: 143-179; Kendall R, Feghali-Bostwick C A, Fibroblasts in fibrosis: novel roles and mediators. Front Pharmacol., 2014; 5: 1-13).

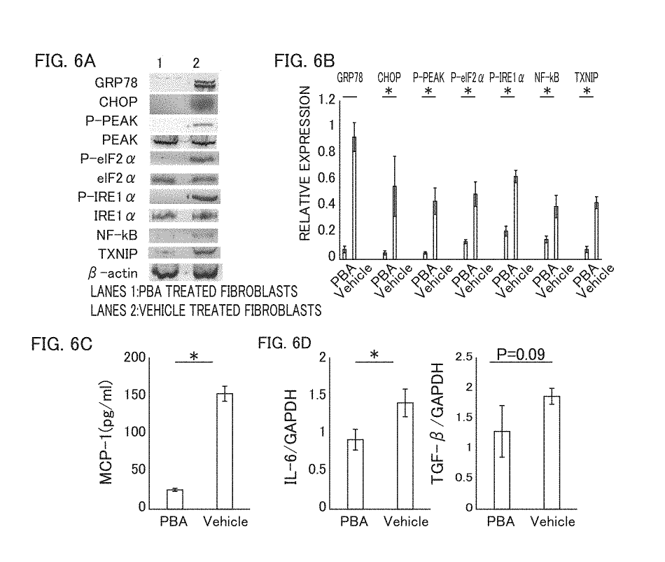

Accordingly, in order to extensively study the correlation between cGVHD-induced endoplasmic reticulum stress and fibroblast dysfunction, fibroblasts from PBA-treated and vehicle-treated mouse lacrimal glands were cultured. FIG. 6A to FIG. 6D show that cGVHD-induced endoplasmic reticulum stress in lacrimal gland fibroblasts was alleviated when endoplasmic reticulum stress was alleviated by PBA. FIG. 6A shows the results of immunoblot analysis of endoplasmic reticulum stress markers, activation markers, and fibrosis markers in mouse lacrimal gland fibroblasts. In FIG. 6A, Lane 1 corresponds to PBA-treated lacrimal gland-derived fibroblasts, and Lane 2 corresponds to vehicle-treated lacrimal gland-derived fibroblasts.

FIG. 6B shows the results of quantitative analysis of the corresponding protein bands (PBA-treated fibroblasts and vehicle-treated fibroblasts). In FIG. 6B, the data is shown as the mean±SD (PBA: n=4, vehicle: n=4, and *P<0.05).

FIG. 6C shows the results from ELISA measurements of the protein levels of MCP-1 produced by PBA-treated fibroblasts and vehicle-treated fibroblasts. In FIG. 6C, the data is shown as the mean±SD (PBA: n=4, vehicle: n=4, and *P<0.05).

FIG. 6D shows the results from qPCR analysis of IL-6 and TGF-β in PBA-treated fibroblasts and vehicle-treated fibroblasts. In FIG. 6D, the values are shown as the mean±SD (PBA: n=4, vehicle: n=4, and *P<0.05).

The results from immunoblot analysis of cultured fibroblasts shown in FIG. 6A clearly show that GRP78, CHOP, phosphorylated PERK, phosphorylated eIF2α, and phosphorylated IRE1α were markedly inhibited by treating mice with PBA as compared with solvent vehicle-treated mice. Therefore, the results shown in FIG. 6A to FIG. 6D clearly indicate that (1) the protein levels of each of HSP47, CTGF, a fibroblast activation marker, and a fibrosis marker; (2) production of MCP-1; and (3) the mRNA levels of IL-6 were decreased in the PBA-treated fibroblasts as compared with the vehicle-treated fibroblasts. That is, the above results show that (1) endoplasmic reticulum stress resulting from cGVHD may activate fibroblasts in a harmful way, thereby inducing extensive fibrosis while (2) PBA can prevent fibroblast dysfunction by alleviating endoplasmic reticulum stress.

[Correlation between Endoplasmic Reticulum Stress and Differentiation of Macrophages into M2 Macrophages]

As described above, macrophages play an important role in immune responses and inflammation. Senescent macrophages are known to be adversely related to the development process of cGVHD. Further, it is suggested that (1) endoplasmic reticulum stress may promote differentiation into M2 macrophages, and (2) M2 macrophages may be involved in fibrosis-related diseases (Oh J, Riek A E, Weng S, et al., Endoplasmic Reticulum Stress Controls M2 Macrophage Differentiation and Foam Cell Formation. The Journal of Biological Chemistry, 2012; 287: 11629-11641;
Xue J, Sharma V, Hsieh M H, et al., Alternatively activated macrophages promote pancreatic fibrosis in chronic pancreatitis. Nature Communications, 2015; 6: 1-11;
Shivshankar P, Halade G V, Calhoun C, et al., Caveolin-1 deletion exacerbates cardiac interstitial fibrosis by promoting M2 macrophage activation in mice after myocardial infarction. J Mol Cell Cardiol., 2014; 76: 84-93).

Accordingly, a possible correlation between cGVHD-induced endoplasmic reticulum stress and macrophage dysfunction (differentiation into M2 macrophages) was extensively studied. First, immunostaining was performed to investigate whether macrophages in a mouse lacrimal duct could express endoplasmic reticulum stress marker CHOP.

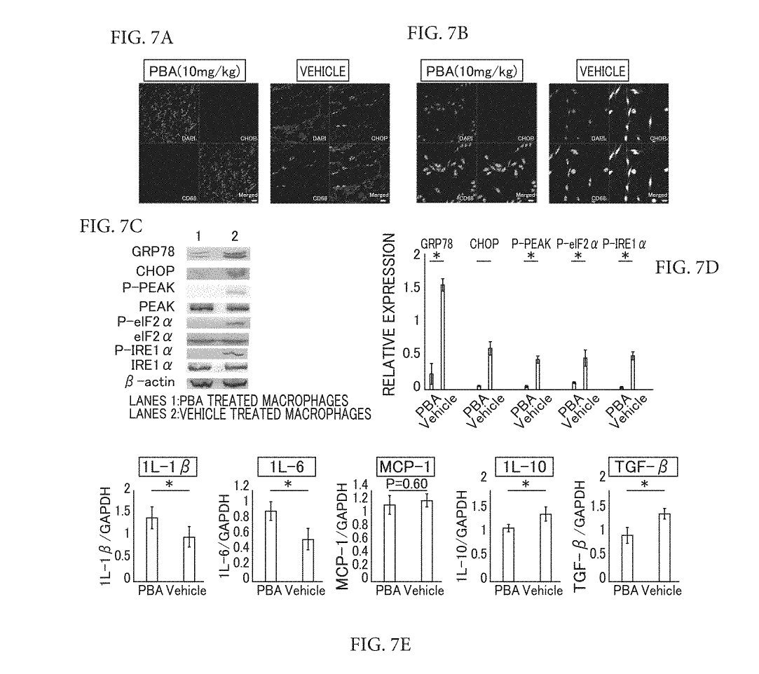

FIG. 7A to FIG. 7E show that endoplasmic reticulum stress resulting from cGVHD in macrophages was alleviated by using PBA. FIG. 7A shows immunofluorescence images of a PBA-injected lacrimal gland and a vehicle-injected lacrimal gland. In FIG. 7A, images were captured at a magnification of ×200. The scale bars indicate 20 μm. Macrophages and CHOP were stained red and green, respectively. As determined from the immunofluorescence images of the lacrimal glands shown in FIG. 7A, CHOP-expressing macrophages were found to migrate to tissues in a solvent vehicle-treated mouse. This indicates that macrophages under endoplasmic reticulum stress are related to the progress of cGVHD. In contrast, such macrophages were not observed in a lacrimal gland collected from a PBA-treated mouse.

Next, in order to obtain a deeper insight about endoplasmic reticulum stress in cGVHD-affected macrophages, splenic macrophages from a PBA-treated mouse and a vehicle-treated mouse were cultured. FIG. 7B shows the results from immunofluorescence of cultured splenic macrophages from the PBA-treated mouse and the vehicle-treated mouse. The images were captured at a magnification of ×200. The scale bars indicate 20 μm. Macrophages and CHOP were stained red and green, respectively. The results from immunohistochemistry shown in FIG. 7B reveal that vehicle-treated splenic macrophages express CHOP in contrast with PBA-treated splenic macrophages.

FIG. 7C shows the results from immunoblot analysis of endoplasmic reticulum stress markers in mouse splenic macrophages. In FIG. 7C, Lane 1 corresponds to splenic macrophages from a PBA-treated mouse, and Lane 2 corresponds to splenic macrophages from a vehicle-treated mouse.

FIG. 7D shows the results from quantitative analysis of the corresponding protein bands. In FIG. 7D, the data is shown as the mean±SD (PBA: n=4, vehicle: n=4, and *P<0.05). The results from immunoblot analysis of macrophages shown in FIG. 7C and FIG. 7D clearly show that endoplasmic reticulum stress markers GRP78, CHOP, phosphorylated PERK, phosphorylated eIF2α, and phosphorylated IRE1α were substantially inhibited by treating macrophages with PBA as compared with solvent vehicle-treated macrophages.

Further, qPCR was performed to investigate the gene expressions of (1) M1 macrophage markers IL-1β, IL-6, and MCP-1 and (2) M2 macrophage markers TGF-β and IL-10 in splenic macrophages (Sene A, Khan A A, Cox D, et al., Impaired Cholesterol Efflux in Senescent Macrophages Promotes Age-Related Macular Degeneration. Cell Metab., 2013; 17: 549-561).

FIG. 7E shows the results from qPCR analysis of M1 macrophage markers and M2 macrophage markers from spleens. In FIG. 7E, the data is shown as the mean±SD (PBA: n=4 to 5, vehicle: n=4 to 5, and *P<0.05). As clearly understood from the results shown in FIG. 7E, the mRNA expressions of M2 macrophage markers TGF-β and IL-10 were found to be increased in the vehicle-treated macrophages as compared with the PBA-treated macrophages. In contrast, the PBA-treated macrophages were found to show higher gene expressions of M1 macrophage markers IL-1β and IL-6 than the solvent vehicle-treated macrophages. The results described above suggest that (1) cGVHD-induced endoplasmic reticulum stress may cause alternative differentiation of macrophages into to an activated phenotype, and (2) cGVHD-induced fibrosis due to transformation of macrophages into an activated phenotype can be alleviated by allowing PBA to reduce endoplasmic reticulum stress in the macrophages.

### <Discussion>

As described above, the results reveal that (1) increased endoplasmic reticulum stress is correlated to (2) increased activities and/or expressions of proinflammatory molecules such as NF-κB and TXNIP in cGVHD-affected organs. For example, the results described above suggest that NF-κB is activated in cGVHD-affected organs, and the activation is in part due to endoplasmic reticulum stress resulting from cGVHD. Non-Patent Documents 6 to 8 listed above describe that (1) TXNIP is produced downstream of the PERK and IRE1α pathways; (2) TXNIP plays an important role at a “branching point” where cells under endoplasmic reticulum stress are destined to recover their homeostasis or undergo apoptosis; and (3) TXNIP induces extensive inflammation by activating inflammasome NOD-like receptor family pyrin domain 3 (NLRP3). However, the correlation between TXNIP and cGVHD has not been known until now. The results as described above reveal that the expression of endoplasmic reticulum stress-induced TXNIP can be correlated with physical disorders due to cGVHD.

FIG. 8 shows a putative correlation between cGVHD and endoplasmic reticulum stress. As shown in FIG. 8, a cGVHD-affected organ will likely suffer from oxidative stress. In response to this, endoplasmic reticulum stress may be increased in epithelial cells, fibroblasts, and macrophages. As a result, (1) inflammation-related molecules such as NF-κB and TXNIP may be expressed and/or activated in an uncontrollable manner; (2) fibroblasts become dysfunctional, resulting in formation of abnormal collagen bundles; and (3) macrophages may be differentiated into the M2 phenotype which may be involved in aberrant fibrosis. Further, endoplasmic reticulum stress is thought to be increased by inflammation, and thus a vicious circle of endoplasmic reticulum stress and inflammation may be formed in a cGVHD-affected organ. Therefore, reduction of endoplasmic reticulum stress resulting from cGVHD may also help to escape from this vicious circle. In summary of the above, PBA can be an effective drug for treating cGVHD by alleviating endoplasmic reticulum stress.

1. A therapeutic or prophylactic agent for graft-versus-host disease, comprising a phenylbutyric acid or a pharmaceutically acceptable salt thereof as an active ingredient.

2. The therapeutic or prophylactic agent according to claim 1, wherein the graft-versus-host disease is a graft-versus-host disease that manifests after bone marrow transplantation.

3. The therapeutic or prophylactic agent according to claim 1, wherein the graft-versus-host disease is ocular graft-versus-host disease.

4. The therapeutic or prophylactic agent according to claim 3, wherein the graft-versus-host disease is dry eye.

5. The therapeutic or prophylactic agent according to claim 1, wherein the graft-versus-host disease is a graft-versus-host disease accompanied by fibrocyte infiltration.

6. The therapeutic or prophylactic agent according to claim 1, wherein the phenylbutyric acid is 4-phenyl-n-butyric acid or a pharmacologically acceptable salt thereof.

7. An agent for inhibiting fibrocyte infiltration, comprising a phenylbutyric acid or a pharmaceutically acceptable salt thereof as an active ingredient.

8. The agent for inhibiting fibrocyte infiltration according to claim 7, wherein the phenylbutyric acid is 4-phenyl-n-butyric acid or a pharmacologically acceptable salt thereof.

9. An agent for inhibiting a decrease in goblet cells, comprising a phenylbutyric acid or a pharmaceutically acceptable salt thereof as an active ingredient.

10. The agent for inhibiting a decrease in goblet cells according to claim 9, wherein the phenylbutyric acid is 4-phenyl-n-butyric acid or a pharmacologically acceptable salt thereof.

11. An agent for inhibiting a decrease in tear secretion, comprising a phenylbutyric acid or a pharmaceutically acceptable salt thereof as an active ingredient.

12. The agent for inhibiting a decrease in tear secretion according to claim 11, wherein the phenylbutyric acid is 4-phenyl-n-butyric acid or a pharmacologically acceptable salt thereof.

13. A method of treating or preventing graft-versus-host disease, comprising administering a phenylbutyric acid or a pharmacologically acceptable salt thereof.

14. The method according to claim 13, wherein the graft-versus-host disease is a graft-versus-host disease that manifests after bone marrow transplantation.

15. The method according to claim 13, wherein the graft-versus-host disease is ocular graft-versus-host disease.

16. The method according to claim 15, wherein the graft-versus-host disease is dry eye.

17. The method according to claim 13, wherein the graft-versus-host disease is a graft-versus-host disease accompanied by fibrocyte infiltration.

18. The method according to claim 13, wherein the phenylbutyric acid is 4-phenyl-n-butyric acid or a pharmacologically acceptable salt thereof.

19. A method of inhibiting fibrocyte infiltration, comprising administering a phenylbutyric acid or a pharmacologically acceptable salt thereof.

20. The method according to claim 19, wherein the phenylbutyric acid is 4-phenyl-n-butyric acid or a pharmacologically acceptable salt thereof.

21. A method of inhibiting a decrease of goblet cells, comprising administering a phenylbutyric acid or a pharmacologically acceptable salt thereof.

22. The method according to claim 21, wherein the phenylbutyric acid is 4-phenyl-n-butyric acid or a pharmacologically acceptable salt thereof.

23. A method of inhibiting a decrease in tear secretion, comprising administering a phenylbutyric acid or a pharmacologically acceptable salt thereof.

24. The method according to claim 23, wherein the phenylbutyric acid is 4-phenyl-n-butyric acid or a pharmacologically acceptable salt thereof.
